(19) United States
(12) Patent Application Publication
(10) Pub. No.: US 2023/0181830 A1
(43) Pub. Date: Jun. 15, 2023

(54) HIGH-PRESSURE MEDICAL INJECTOR

(71) Applicant: MEDTRON AG, Saarbrucken (DE)

(72) Inventors: Stefan Kreber, Saarbrucken (DE); Michael Schweigmann, Zweibruken (DE); Hanno Altmeyer, Neunkirchen (DE)

(21) Appl. No.: 17/993,265

(22) Filed: Nov. 23, 2022

(30) Foreign Application Priority Data
Nov. 25, 2021 (EP) 21210433.5

(51) Int. Cl. A61M 5/20; A61M 5/00; A61M 5/44

(52) U.S. Cl. CPC A61M 5/2053; A61M 5/007; A61M 5/008; A61M 5/44

(57) ABSTRACT

The invention relates to a medical high-pressure injector for injecting a liquid provided in a syringe (2.1, 2.2) into a patient, wherein the injector has at least one syringe holder (10) with an associated drive unit for the syringe (2.1, 2.2) held therein as well as connecting lines for transmitting the liquid from the syringe (2.1, 2.2) into a patient tube and/or for filling the syringe (2.1, 2.2) that is held in the syringe holder (10), wherein the connecting lines are embodied in an interchangeable control cassette (3) and the injector has a cassette holder (11) with a receiving chamber (110) that can be opened and closed, into which the control cassette (3) can be inserted.

### BACKGROUND OF THE INVENTION

### Field of the Invention

This invention relates to a medical high-pressure injector for injecting a liquid provided in a syringe into a patient, wherein the injector has at least one syringe holder with an associated drive unit for the syringe held therein as well as connecting lines for transmitting the liquid from the syringe into a patient tube and/or for filling the syringe that is held in the syringe holder.

### Discussion of Related Art

Medical high-pressure injectors of the type mentioned above are known and are used, for example, in the context of computed tomography (CT) or magnetic resonance tomography (MRT) in order to inject a contrast medium and/or other liquids such as saline solution into the patient at a high pressure of up to 83 bar as part of a corresponding medical imaging examination. An example of such a medical high-pressure injector is disclosed in German Patent Reference DE 10 2004 032 970 A1. The syringes that are held in the syringe holder of the injector, usually one or two such syringes, are connected via a system of connecting lines, which include a pressure line for injecting the at least one liquid from the syringe into the patient and also include at least one suction line, which, before the injection into the patient, makes it possible to fill the syringe with the corresponding liquid from a corresponding reservoir. The flow directions that are desired when filling each syringe and when injecting the liquid into the patient are ensured by correspondingly provided check valves. An example of such a system of connecting lines is the subject of German Patent Reference DE 203 01 094 U1. Due to prevailing hygiene requirements, such connecting lines are single-use articles and must be laboriously replaced and connected by correspondingly trained specialists before the examination starts, which is labor-intensive and error-prone. In this context, special care must be taken that no air or accumulations of other gases are contained in the connecting lines or syringes since injecting gas bubbles into the patient would potentially result in life threatening complications. In addition, the connecting lines that have been used before now always define a correspondingly large volume, which must be filled with liquid and is discarded together with the connecting lines after the examination is completed, which seems to be in need of improvement. Passive valves are also used, which do not make it possible to generate an admission pressure in the liquid for bolus sharpening purposes.

### SUMMARY OF THE INVENTION

One object of this invention is to provide a medical high-pressure injector of the type mentioned above but in a way that overcomes the disadvantages of the prior art.

In order to attain the stated object and others this invention proposes a medical high-pressure injector according to the features described in this specification and in the claims, including the dependent claims.

The proposal according to this invention provides for the connecting lines to be embodied in an interchangeable control cassette and for the injector to have a corresponding cassette holder with a receiving chamber that can be opened and closed, into which the control cassette can be interchangeably inserted.

According to this invention, the connecting lines, at least over a larger segment thereof, are ideally embodied entirely inside an interchangeable control cassette that can be inserted into a corresponding cassette holder of the high-pressure injector so that the effort required to connect the connecting lines is reduced to a minimum. According to this invention, all that is required is for such a control cassette, which has the connecting lines, to be inserted into the cassette holder provided in the injector and for the required number of syringes to be connected and also for the patient tube to be connected to the control cassette. If need be, the control cassette can also be connected to reservoirs of the injection liquids at corresponding connections in order draw the liquid into the syringes in an intrinsically known way before the examination.

To accomplish this, the control cassette is preferably embodied with different connections, which can be embodied, for example, as part of a Luer lock connection.

According to this invention, the connecting lines that are required for the operation of the medical high-pressure injector are embodied as at least partially, preferably entirely, inside the interchangeable control cassette so that they are embodied in the control cassette in a preconfigured way with the shortest possible length and corresponding minimal volume and after the connection of the syringes, the patient tube, and optionally the reservoirs, an immediate, complete, and correct connection situation is provided for the safe operation of the high-pressure injector according to this invention. This thus significantly reduces the effort required for training and for connecting the connecting lines and also reduces the risk of operator error.

According to one embodiment of this invention, the control cassette includes an at least two-part construction and has a hard part and at least one diaphragm. In the hard part, a surface embodied as a routing surface is provided with line segments for forming the connecting lines in the control cassette so that they are milled into the surface in groove-like fashion, for example, or are embodied in the form of groove-like recesses in it that are produced directly during the shaping of the hard part. The surface of the hard part that is provided with the line segments in this way and is referred to as a routing surface is bordered by a diaphragm that is mounted on it in a liquid-tight way, such as the diaphragm forms a liquid-tight closure of the line segments embodied in the hard part and covers the routing surface including the line segments provided therein.

According to another embodiment of this invention, on the surface of the hard part, which is on the opposite side from the routing surface and is referred to as the valve surface, the hard part has valve segments of the connecting lines, which are provided, preferably in the form of groove like recesses, in the valve surface. With an additional diaphragm that is mounted on the valve surface in a liquid-tight way, the valve segments are also covered in a liquid-tight way and, via connecting bores extending through the hard part from the routing surface to the valve surface, communicate with the line segments in the region of the routing surface. Together with the line segments, the valve segments form the connecting lines in the control cassette.

In addition, the diaphragm that covers the valve segments can be elastically deflected by a force of pressure that is exerted from the outside of the diaphragm so that it can serve as a valve and leaves the flow conduit open in the relaxed state, but narrows or completely closes the flow conduit when a force of pressure is exerted. To accomplish this, according to this invention the cassette holder is equipped with or has plungers that can be slid forward into the receiving chamber and that are respectively associated with valve segments of an inserted control cassette. With associated linear drive units, a corresponding control unit can slide the plungers forward into the receiving chamber or retract them from the receiving chamber in the opposite direction. With a plunger that has been slid forward into the receiving chamber and is pressing against a valve segment, it is possible to selectively at least partially or also completely close the respective valve segment as a function of the force of pressure exerted and, after the plunger is retracted in the opposite direction, to leave the respective valve segment completely open. In this way, it is possible for not only the connecting lines, but also with the elastically deflectable diaphragm over the valve segments for the valves that are required for the flow control to be embodied in the control cassette of the medical high-pressure injector that is provided according to this invention and as a result for the check valves, which have been provided in the connecting lines before now, to be replaced and be provided in the control cassette.

According to another embodiment of this invention, the cassette holder of the high-pressure injector according to this invention is embodied and dimensioned so that it forms a receiving chamber for the control cassette, which chamber can be closed by a door and into which the control cassette can be inserted in a form-fitting way. In this respect, the receiving chamber is embodied as a recess that is adapted to the contour of the control cassette and comprises a floor, side walls, and the door, which is spaced apart from the floor by the side walls and is for opening and closing the receiving chamber. The plungers for manipulating the valve segments of the control cassette are inserted in the region of the floor and can be slid forward into the receiving chamber. The control cassette is thus inserted into the receiving chamber together with the valve surface ahead of time so that the valve surface and the diaphragm mounted on it come into contact with the floor and the plungers positioned therein. After the door is closed, it comes to rest on the opposite side against the routing surface of the control cassette and the diaphragm mounted thereon. Since the closed receiving chamber is filled by the control cassette in a form-fitting way, the diaphragm of the valve surface against the floor of the receiving chamber and the diaphragm of the routing surface against the closed door are provided with a pressure-proof support so that the control cassette can be acted on with the liquid pressures of up to 83 bar that are typical for this application without the occurrence of leakage problems.

According to another embodiment of this invention, the hard part is made, for example, of an injection molded plastic and the diaphragms are each made of a plastic film that can be welded to the hard part so that the desired liquid-tight connection between the hard part and the diaphragm can be produced, for example with a laser weld. With such embodiment, the control cassette according to this invention can be efficiently produced in large quantities, which particularly takes into account the fact that the control cassette, which contains the valves and connecting lines, as a single-use part, must be replaced and discarded after completion of an examination cycle of the high-pressure injector.

Incidentally, the use of the control cassette as a single-use part includes both a one-time use with only one patient and also a use for a limited period of time, for example a day or 24 hours for a corresponding number of patients.

It goes without saying that if need be, line segments can also be embodied in the valve surface and valve segments can also be embodied in the region of or near the routing surface.

In order to be able to produce the valve function in the control cassette with the elastically deflectable diaphragm, the plungers that can be slid forward into the receiving chamber, for example, can be actuated pneumatically or by electric linear drive units, and are positioned in the cassette holder relative to the installation position of the control cassette so that they can be pressed against an associated valve segment of the control cassette that is inserted into the receiving chamber, producing a force of pressure against a desired position of the control cassette. Depending on the path of the line segments and valve segments in the control cassette, the valves can be provided at suitable positions in the control cassette through a corresponding positioning of the plungers.

According to another embodiment of this invention, the connecting lines embodied in the control cassette can also comprise a device for separating gas bubbles from a liquid flowing through the connecting lines in order to inhibit the inadvertent introduction of a gas volume into the patient tube and finally into the patient.

Such a device for separating gas bubbles, which can be integrated into the control cassette, can for example, comprise a conduit segment of the connecting line, which extends in a circular arc shape and whose radius extends in a vertically oriented plane, wherein a gas outlet opening is provided at the highest point of the region and in addition, the conduit tapers in the flow direction of the liquid. Such a device permits a reliable separation of any gas volumes that accumulate in the liquid flow. In particular, the device for separating the gas bubbles is integrated into the sequence of line segments situated one after another in the flow direction through the control cassette, such as a line segment communicates with an inlet of the conduit of the device while the outlet of the conduit is adjoined by another line segment, wherein at both the inlet end and the outlet end, a corresponding valve segment can be provided in order to control the flow.

According to another embodiment of this invention, the control cassette can also comprise an optical detection device for detecting gas bubbles in a line segment, preferably in the line segment that leads to the patient tube.

According to another embodiment of this invention, such a detection device can have two deflecting prisms positioned on opposite sides relative to the connecting line that is to be monitored, one of which is illuminated by a light source positioned in the wall of the receiving chamber and guides the light beam through the monitored line segment to the opposing deflecting prism and the latter deflects the incident light beam to at least one sensor positioned in the wall of the receiving chamber. The deflecting prisms can, for example, be embodied integrally in the hard part in the course of or during the latter's manufacture so that they are located at a defined position of the control cassette and cooperate in a beam path with corresponding light sources and sensors, which are positioned in stationary fashion in the receiving chamber of the high-pressure injector according to this invention. Thus according to this invention, when the control cassette is inserted into the receiving chamber, this simultaneously produces the correct positioning of the deflecting prisms relative to the light source and sensors.

According to another embodiment of this invention, the control cassette can be equipped in some regions with a heating device for the liquid that is conveyed through the connecting lines. Such a heating device for heating the liquid that is conveyed through the connecting lines makes it possible on the one hand to heat the liquid to a temperature that is comfortable for the patient, in particular the patient's body temperature, and on the other hand, it is possible for contrast medium, which is frequently relatively viscous at room temperature, to be heated to a temperature that is suitable for desired high flow rates.

According to one embodiment of this invention, the heating device can for example be formed by a radiation-absorbing coating that is provided in some regions of the control cassette and an associated light source in the receiving chamber, for example a corresponding LED spotlight.

For this purpose, one or more line segments in the control cassette can be routed with a meandering path and a correspondingly colored film can be applied to one of the diaphragms, for example, in a predetermined sector, or the hard part can also be correspondingly colored. In addition to the preheating of the liquid that is to be injected, the embodiment of the heating device with a spotlight and a radiation-absorbing coating also has the advantage that the switching on and off of the spotlight can produce a rapid fine adjustment of the temperature without hysteresis.

According to another embodiment of this invention, the control cassette and at least one syringe, preferably two syringes, are combined into an interchangeable module and are connected to one another so that they can be jointly inserted into the receiving chamber and the syringe holder. Such a fixed combination of the syringe(s) and control cassette simplifies the use of the high-pressure injector according to this invention even further since it is no longer necessary to connect the control cassette to each of the two syringes. Depending on the syringe type, the syringes can also be inserted into the receiving chamber of the high-pressure injector according to this invention in either a vertical or horizontal orientation.

According to another embodiment of this invention, the correct insertion of the control cassette into the receiving chamber of the high-pressure injector according to this invention can be ensured by the fact that the receiving chamber and/or the control cassette is equipped with or has means for a correctly positioned insertion of the control cassette into the receiving chamber. This can be achieved, for example, by means of or with protruding pins provided in the receiving chamber, which engage with corresponding recesses of the control cassette if the latter is inserted into the receiving chamber in the correct orientation. It goes without saying that this arrangement of pins and recesses can also be reversed if so desired.

Finally, according to another embodiment of this invention, the control cassette of the high-pressure injector according to this invention can also be embodied with identification means, which are readable from the high-pressure injector when a control cassette is inserted into the receiving chamber. Examples of such identification means include a microchip on the control cassette, a PIN code, a barcode, an RFID chip, and the like. Such an identification means can be used to present not only proof of authenticity, but also expiration dates, durations of use, injection parameters, and the like or this information can be stored in the identification means.

### BRIEF DESCRIPTION OF THE DRAWINGS

Other embodiments and details of this invention will be explained below based on the drawings, which show an exemplary embodiment in the drawings, wherein:

FIG. 1 shows a control cassette of a high-pressure injector according to one embodiment of this invention in an exploded view;

FIG. 2 shows the front view of the hard part of the control cassette according to FIG. 1;

FIG. 3 shows the back view of the hard part of the control cassette according to FIG. 1;

FIG. 4 shows the control cassette that is inserted into the high-pressure injector according to this invention;

FIG. 5 shows the arrangement according to FIG. 4 in a cutaway side view;

FIG. 6a shows the top view of a detection device according to FIG. 2, in an enlarged depiction; and

FIG. 6b shows the detection device according to FIG. 6a from a perspective that is rotated by 90°.

### DETAILED DESCRIPTION OF THE INVENTION

The figures show a medical high-pressure injector, for example, for use in combination with a CT or MRT device, which according to the depiction in FIG. 4 comprises a syringe mount 10 for two syringes 2.1 and 2.2 along with corresponding drive units, not shown here, for syringe actuation and a corresponding control unit. The syringe 2.1 is or can be filled, for example, with a contrast medium and the syringe 2.2 is or can be filled with saline solution.

The respective syringe openings of the syringes 2.1 and 2.2 are connected to corresponding connections 36.1, 36.2 of a control cassette 3, which is in turn inserted, preferably in a form-fitting way, into a corresponding receiving chamber 110 of a cassette holder 11 of the high-pressure injector. The receiving chamber 110 thus directly adjoins the syringe mount 10

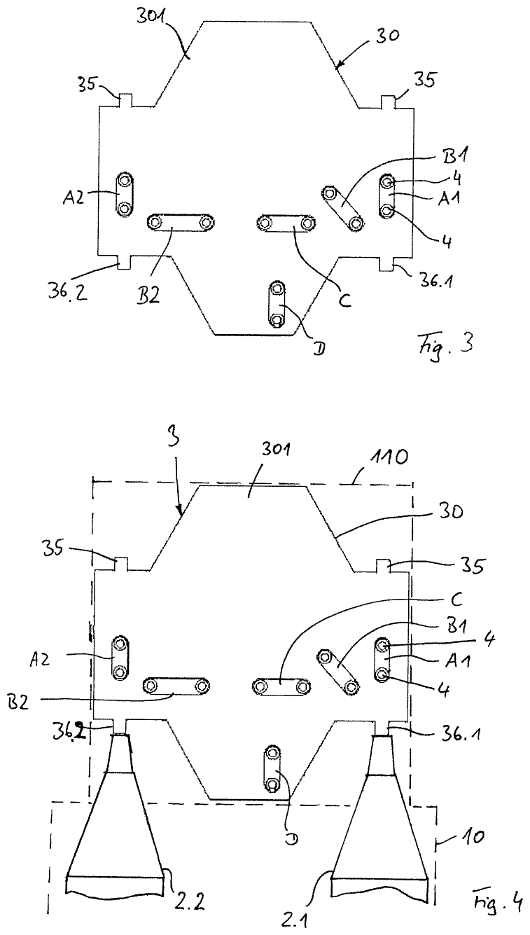

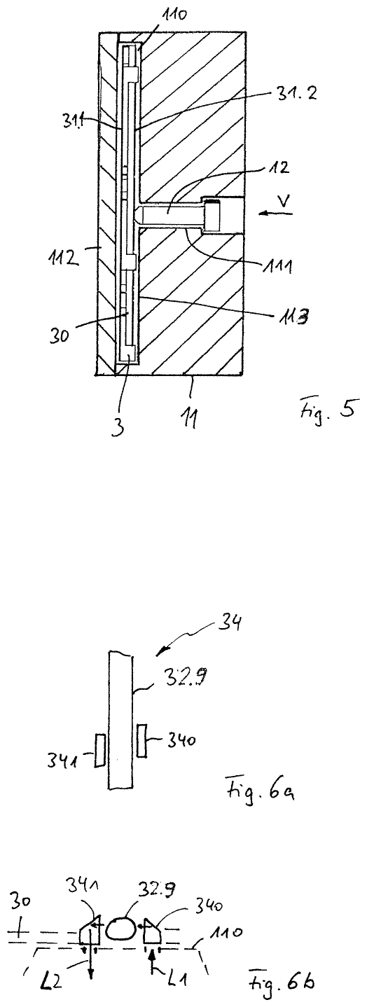

As is particularly clear from the depiction in FIG. 5, the receiving chamber 110 embodied in the cassette holder 11 can be closed with a corresponding door 112, wherein the dimensioning of the receiving chamber 110 is selected so that the control cassette 3 can be inserted into the receiving chamber 110 only in a predetermined orientation and after the door 112 is closed, is supported at the front against the door 112 and at the back against the wall and/or floor 113 of the receiving chamber 110. The door 112 can likewise cover and close the syringe mount 10. The orientation is selected so that the surface of the control cassette 3 that is visible in FIG. 2 rests against the door 112 and the surface of the control cassette 3 that is visible in FIG. 3 rests against the wall and/or the floor 113 of the receiving chamber 110.

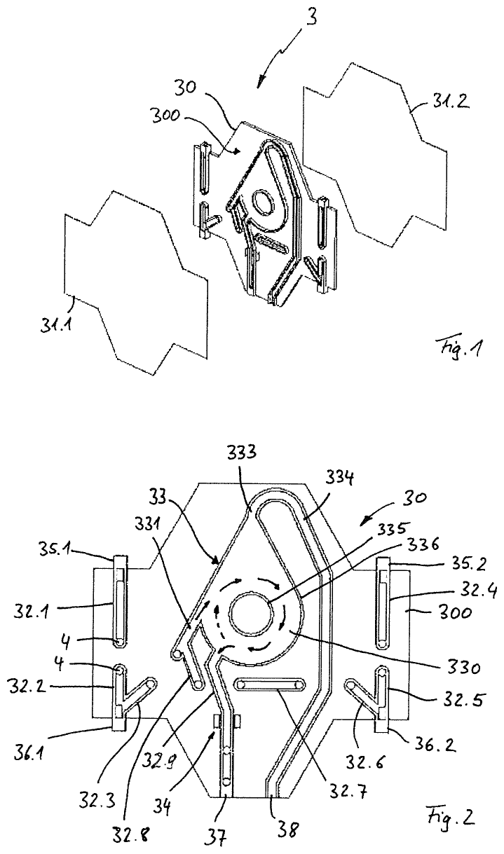

In the way that will be described in greater detail below, all of the connecting lines for the operation of the high-pressure injector are embodied inside the control cassette 3, which is shown in greater detail in FIGS. 1-3.

The control cassette 3 has a multi-part construction and comprises a centrally located hard part 30, for example, composed of injection-molded plastic, each of whose two opposing surfaces has a respective diaphragm 31.1, 31.2 made of an elastically stretchable plastic film placed against it, which is welded to the hard part 30 in a liquid-tight way, for example, by means of or with laser welding.

As is particularly clear from the depiction in FIG. 2, in the region of its surface that is covered by the diaphragm 31.1, which is referred to below as the routing surface 300, the hard part 30 is provided with a plurality of groove-like line segments 32.1, 32.2, 32.3, 32.4, 32.5, 32.6, 32.7, 32.8, 32.9 as well as a device 33 for separating gas bubbles, which will be explained below.

When the hard part 30 is made of an injection-molded plastic, these line segments 32.1, 32.2, 32.3, 32.4, 32.5, 32.6, 32.7, 32.8, 32.9 and/or the device 33 can be incorporated directly through a corresponding design of the injection mold used. Alternatively, they can also be machined out of a solid material, for example, by means of or with cutting.

In the region of the outer circumference of the hard part 30 in addition to the above-mentioned connections 36.1 and 36.2 for connecting the syringes 2.1 and 2.2, other connections 35.1 and 35.2 are visible, which are shown at the opposite edge and, like the connections 36.1 and 36.2, can be embodied as part of a luer lock connector.

The connections 35.1 and 35.2 are used for connecting the control cassette 3 or more specifically its hard part 30 to supply lines of reservoirs, not shown, for contrast medium and saline solution in order to be able to fill the syringes 2.1 and 2.2 that can be connected to the connecting elements 36.1 and 36.2.

The control cassette 3 is inserted into the receiving chamber 110 of the cassette socket 11 and oriented in such a way that when the door 112 is closed, the routing surface covered by the diaphragm 31.1 rests and is supported against it in a pressure-tight way.

On the surface opposite from the routing surface 300, which is shown in greater detail in FIG. 3 and is referred to below as the valve surface 301, groove-like recesses are likewise provided at defined positions, which form valve segments A1, A2, B1, B2, C, and D and, via bores 4 respectively embodied in the end region of the valve segments A1, A2, B1, B2, C, D and extending through the hard part 30 from the valve surface 301 to the routing surface 300 on the opposite side, communicate with line segments (32.1, 32.2, 32.3, 32.4, 32.5, 32.6, 32.7, 32.8, 32.9) there that likewise end at the bores 4 and in this respect, jointly form the connecting lines in the control cassette 3. When the control cassette 3 is inserted into the receiving chamber 110 of the cassette socket 11, the valve surface 301 with the diaphragm 31.2 mounted on it rests in a pressure-tight way against the wall and/or the floor 113 of the receiving chamber.

In order to fill the syringes 2.1 and 2.2 that are connected to the connections 36.1 and 36.2, a respective connecting line is formed starting from the connecting elements 35.1 and 35.2. From the reservoir, not shown, the liquid that is present at the connection 35.1 travels first via the flow conduit 32.1 and the bore 4 provided at the end thereof to the valve surface 301 according to FIG. 3 on the opposite side, and into the conduit A1 embodied therein. From there, another bore 4 leads back to the routing surface 300, into a flow conduit 32.2, and from there, via the connecting element 36.1 into the associated syringe 2.1. Liquid that is present at the connection 35.2 likewise travels via a flow conduit 32.4 and a bore 4 provided at the end into the valve surface 301 and a valve segment A2 there, and via another bore 4 back to the routing surface 300 and the valve segments 32.5 there, which finally leads to the connection 36.2 for the syringe 2.2. In this respect, the syringes 2.1 and 2.2 can be filled with the respectively desired liquid through the control cassette 3 via the above-explained connecting lines embodied therein.

The actual injection procedure of the high-pressure injector from the syringes 2.1 and 2.2 in the direction of the patient takes place via a patient tube, not shown here, which is connected to a corresponding port 37 of the control cassette 3 in the hard part 30. From the connecting element 36.1, the liquid that is to be injected from the syringe 2.1, for example, contrast medium, travels first via a flow conduit 32.3 and the valve segment B1 formed in the valve surface 301 on the opposite side of the hard part 30 and into an inlet 331 of the device for gas separation 33 that is explained in greater detail below, which the liquid then exits via the line segment 32.9 in the direction of the port 37 provided for the patient tube.

Likewise, the liquid that is to be injected from the syringe 2.2 can be transported via the associated connecting element 36.2 and the line segments 32.6 to the valve segment B2 embodied in the valve surface 301 on the opposite side of the hard part 30, and into the line segment 32.7 and then delivered via the valve segment C once again embodied on the valve surface 301 on the opposite side of the hard part 30 and into the line segments 32.8 and from there into the intake 331 of the device 33 in order to finally arrive via the line segment 32.9 at the port 37 to which the patient tube is connected.

The respective alternation of the liquid from the one surface of the hard part according to FIG. 2 to the other surface of the hard part according to FIG. 3 respectively takes place via the through bores 4 that pass through the hard part 30 and that communicate with the line segments 32.1, 32.2, 32.3, 32.4, 32.5, 32.6, 32.7, 32.8, 32.9 in the region of or near the routing surface 330 and with the valve segments H1, H2, B1, B2, C, and D in the region of or near the valve surface 301 on the opposite side.

In order to predetermine the flow of liquids through the control cassette 3 in the above-described way, the respectively desired connecting lines must be opened and the rest of the connecting lines that are not needed must be closed. For example, when filling the syringe 2.1, the valve segment A1 is opened whereas the valve segment B1 that leads in the direction of the port 37 for the patient tube is closed. On the other hand, when injecting liquid from the syringe 2.1 in the direction of the port 37, the valve segment B1 is opened and the valve segment A1 is closed in order to close off the flow path to the connection 35.1. The same is also true for the connecting lines between the connections 35.2 and 36.2

As is particularly clear from the depiction in FIG. 5, this is accomplished by inserting the control cassette 3 into the corresponding cassette holder 11 of the high-pressure injector, which has the receiving chamber 110 for the control cassette 3 and can be closed by means of or with the door 112 after the insertion of a control cassette 3. The diaphragms 31.1 and 31.2 that are mounted in the region of the two surfaces of the hard part 30 of the control cassette 3 are thus provided with support over their entire area along the wall and/or the floor 113 of the receiving chamber 110 and along the inner surface of the door 112 so that they are also able to withstand high pressures of the liquid that is conveyed in the connecting lines of the hard part 30.

In addition, multiple plungers 12 are positioned in corresponding bores 111 in the wall and/or the floor 113 of the cassette holder 11 and can be pressed, for example pneumatically, in the direction of arrow V in FIG. 5 against the adjacent diaphragm 31.2, which covers the valve surface 301 of the hard part 30 shown in FIG. 3, in which the valve segments A1, A2, B1, B2, C, and D are embodied.

Each of these plungers 12 is positioned so that when the control cassette 3 is inserted into the receiving chamber 110 correctly, the plunger is positioned exactly over one of the above-mentioned valve segments A1, A2, B1, B2, C, or D so that with an advancing of the plunger 12 in the direction of arrow V, the flow cross-section through the respective valve segment A1, A2, B1, B2, C, or D can be changed from an open position into a position that narrows or closes this valve segment in that the force of pressure coming from the plunger 12 elastically deflects the adjacent diaphragm 31 in the direction of the hard part 30. In this way, the valves can be embodied to control the flow through the individual connecting lines.

As explained above, the liquid that is injected from the syringe 2.1 or 2.2, on its way through the control cassette 3, flows from the line segments 32.3 and into the valve segment B1 and via the line segments 32.6, 32.7, and 32.8 with the interposition of the valve segments B2 and C through a joint intake 331 into the device 33 for separating gas bubbles, which is integrated into the path of the connecting lines formed by the line segments 32.1, 32.2, 32.3, 32.4, 32.5, 32.6, 32.7, 32.8, 32.9 and the valve segments A1, A2, B1, B2, C, or D. The separation of gas bubbles form the liquid flowing through is achieved by the fact that starting from the intake 331, this liquid is forced into a circular arc-shaped conduit segment 330, which is defined by an outer partition wall 336 and an inner partition wall 335 and also tapers in its internal height in the flow direction depicted with arrows. As a result of this, already upon deflection of the liquid flow supplied from the intake 331 into the circular arc-shaped path 330, large entrained gas bubbles separate out and exit the device 33 via a gas outlet opening 333 situated at the highest point. In this respect, they are discharged separately from the liquid via a discharge line 334 and, via a port 38, are delivered to a corresponding collecting device that is not shown here.

Smaller gas bubbles, however, collect primarily in the region of the circular inner surface 335 and gradually rise from there likewise in the direction of the gas outlet 333 situated at the highest point, and can be discharged separately from the liquid.

The liquid that has been freed of gas influxes, however, exits the device 33 via the line segment 32.9 serving as an outlet in the direction of the patient tube that is connected to the port 37. The line segment 32.9 can be closed or opened by means of or with a valve segment D with a corresponding plunger 12 that is integrated into the flow path, likewise in the region of the valve surface on the opposite side.

In the context of this invention, a particular embodiment of the device 33 permits a particularly reliable separation of any gas bubbles that are contained in the liquid to be performed, wherein the separating action is even greater the higher the flow velocity of the liquid is. The flow velocities in a high-pressure injector are usually particularly high given the operating pressures of up to 83 bar that are present. In addition, partial quantities of the liquid that has been forced on the circular path may not pass directly into the outlet 332, instead executing one or optionally also several additional revolutions through the device 33 on the circular path. This is harmless, however, and only increases the separating action.

To further increase the safety when it comes to undesirable gas inclusions in the liquid that is supplied to the patient tube, the line segment 32.9 is also provided with a detection device 34, which is shown in greater detail in FIGS. 6a and 6b.

This detection device 34 comprises two deflecting prisms 340, 341 positioned on both sides of the preferably transparently embodied line segment 32.9. A light source positioned in the region of the door 112 or the wall 113 of the receiving chamber 110 sends a light beam L1 through a corresponding opening into the deflecting prism 340, which directs the light beam rotated by 90° through the line segment 32.9 that is to be monitored and onto the other deflecting prism 341 in the region of the opposite side of the line segment 32.9. From there, the light beam is directed, once again rotated by 90°, through an opening in the receiving chamber 110 as light beam L2 to corresponding sensors positioned in the cassette holder 11, thus enabling a monitoring of the line segment 32.9. A specific evaluation can be made possible by providing multiple sensors and/or diodes. As soon as a gas bubble flows through the line segment 32.9 instead of liquid, the refractive index changes, which can be reliably detected by the sensors and results, for example, in an immediate closing of the high-pressure injector through closure of the valve segment D.

One advantage of the arrangement shown is that the deflecting prisms 340, 341 can be embodied in a defined position just like the line segments 32.1, 32.2, 32.3, 32.4, 32.5, 32.6, 32.7, 32.8, 32.9 on the routing surface 300 of the hard part 30 and can cooperate with corresponding light sources and sensors positioned in stationary fashion in the cassette holder 11 as soon as the control cassette 3 is inserted into the cassette holder 11 or more specifically the receiving chamber 110 thereof, in the desired, correct orientation. This can be ensured, for example, by means of or with corresponding pins and receiving bores, not shown here, which engage with one another only with a correct orientation of the control cassette 3.

Because in addition, the outlet openings for the light beam L1 and the inlet opening for the light beam L2 are positioned in the door 112 or wall 113 of the receiving chamber 110, they can be easily cleaned, for example, when changing the control cassette 3.

With the above-explained high-pressure injector, by integrating all of the connecting lines as well as a device 33 for separating gas bubbles and a detection device 34 into an interchangeable control cassette 3, it is possible to enormously reduce the changeover times of the high-pressure injector and the amount of effort required to connect the connecting lines and to reliably eliminate error sources.

In addition, after the door 112 associated with the receiving chamber 110 is opened, the control cassettes 3 can be easily removed and exchanged for a new control cassette 3 so that the requirements for embodying the connecting lines as single-use parts can be taken into account.

It goes without saying that instead of the embodiment of the high-pressure injector with two syringes 2.1, 2.2 as shown in the figures, different embodiments with only one syringe or also more than two syringes can also be provided.

In particular, it is possible for the syringes 2.1, 2.2 to be prefabricated as a unit together with the control cassette 3 and in this respect, for them to be pre connected to the connecting elements 36 and possibly even for the syringes 2.1 and 2.2 to be provided as already pre-filled so that it is also no longer necessary to produce any connections with the connecting elements 35. In such case, all that is needed to make the injector ready for operation is for the unit composed of the syringes 2.1, 2.2 and control cassette 3 to be inserted into the combined cassette holder 11 and syringe holder 10 and for the patient tube to be connected to the port 37. If the patient tube is also already pre-connected to the control cassette 3, the latter manual step can also be eliminated and the procedure can be reduced to inserting the pre-connected unit composed of the control cassette 3, the syringes 2.1 and 2.2, and the patient tube.

While in the foregoing specification this invention has been described in relation to certain preferred embodiments, and many details are set forth for purpose of illustration, it will be apparent to those skilled in the art that this invention is susceptible to additional embodiments and that certain of the details described in this specification and in the claims can be varied considerably without departing from the basic principles of this invention.

1-13. (canceled)

14. A medical high-pressure injector for injecting a liquid provided in a syringe (2.1, 2.2) into a patient, wherein the injector has at least one syringe holder (10) with an associated drive unit for the syringe (2.1, 2.2) held therein as well as connecting lines for transmitting the liquid from the syringe (2.1, 2.2) into a patient tube and/or for filling the syringe (2.1, 2.2) that is held in the syringe holder (10), the high-pressure injector comprising the connecting lines embodied in an interchangeable control cassette (3) and the injector having a cassette holder (11) with a receiving chamber (110) that can be opened and closed, into which the control cassette (3) can be interchangeably inserted.

15. The high-pressure injector according to claim 14, wherein the control cassette (3) comprises an at least a two-part construction with a hard part (30) and at least one diaphragm (31.1, 31.2), wherein line segments (32.1, 32.2, 32.3, 32.4, 32.5, 32.6, 32.7, 32.8, 32.9) of the connecting lines are provided in a surface of the hard part (30) embodied as a routing surface (300) and a diaphragm (31.1) is mounted on the routing surface (300) in a liquid-tight way and covers the line segments (32.1, 32.2, 32.3, 32.4, 32.5, 32.6, 32.7, 32.8, 32.9) in the hard part (30).

16. The high-pressure injector according to claim 15, wherein in the surface of the hard part (30) that is on the opposite side from the routing surface (300) and is embodied as a valve surface (301) and is covered by a diaphragm (31.2), which is mounted on the valve surface (301) in a liquid-tight way, valve segments (A1, A2, B1, B2, C, D) of the connecting lines communicate with the line segments (32.1, 32.2, 32.3, 32.4, 32.5, 32.6, 32.7, 32.8, 32.9) via connecting bores (4), which pass through the hard part (30) from the routing surface (300) to the valve surface (301), and jointly form the connecting lines, and the cassette holder (11) is equipped with plungers (12), which are associated with the valve segments (A1, A2, B1, B2, C, D) of the inserted control cassette (3) and can be slid forward into the receiving chamber (110) in order to exert a force of pressure on the diaphragm (31.2) in the region of the associated valve segment (A1, A2, B1, B2, C, D) and to at least partially close the valve segment (A1, A2, B1, B2, C, D).

17. The high-pressure injector according to claim 16, wherein the hard part (30) of an injection-molded plastic and the diaphragm (31.1, 31.2) is made of a plastic film that can be welded to the hard part (30).

18. The high-pressure injector according to claim 17, wherein the connecting lines embodied in the control cassette (3) comprise a device (33) for separating gas bubbles from a liquid flowing through the connecting lines.

19. The high-pressure injector according to claim 18, wherein the device (33) for separating the gas bubbles comprises a conduit (330), extending in a circular arc shape and communicating with line segments (32.1, 32.2, 32.3, 32.4, 32.5, 32.6, 32.7, 32.8, 32.9) of the connecting lines and whose radius extends in a vertically oriented plane, and wherein a gas outlet opening (333) is provided at the highest point of the device (33) and the conduit (330) tapers in the flow direction of the liquid.

20. The high-pressure injector according to claim 19, wherein the control cassette (3) comprises an optical detection device (34) for detecting gas bubbles in a line segment (32.1, 32.2, 32.3, 32.4, 32.5, 32.6, 32.7, 32.8, 32.9).

21. The high-pressure injector according to claim 20, wherein the detection device (34) has two deflecting prisms (340, 341) positioned on opposite sides relative to a line segment (32.1, 32.2, 32.3, 32.4, 32.5, 32.6, 32.7, 32.8, 32.9), wherein one deflecting prism (340) is associated with a light source positioned in the wall of the receiving chamber (110) and by this deflecting prism (340), the light beam can be guided through the line segment (32.1, 32.2, 32.3, 32.4, 32.5, 32.6, 32.7, 32.8, 32.9) to the opposing deflecting prism (341) and the deflecting prism (341) is associated with a sensor positioned in the wall of the receiving chamber (110).

22. The high-pressure injector according to claim 21, wherein in certain regions, the control cassette (3) is equipped with a heating device for the liquid that is conveyed through the connecting lines.

23. The high-pressure injector according to claim 22, wherein the heating device is formed by a radiation-absorbing coating that is provided in some regions of the control cassette (3) and an associated light source in the receiving chamber (110).

24. The high-pressure injector according to claim 23, wherein the control cassette (3) and at least one syringe (2.1, 2.2) are combined into an interchangeable unit and connected to each other and can be jointly inserted into the receiving chamber (110) and the syringe holder (10).

25. The high-pressure injector according to claim 24, wherein the receiving chamber (110) and/or the control cassette (3) is each equipped with a correctly positioned insertion of the control cassette (3) into the receiving chamber (110).

26. The high-pressure injector according to claim 25, wherein the control cassette (3) is embodied with an identification which is readable from the high-pressure injector when a control cassette (3) is inserted into the receiving chamber (110).

27. The high-pressure injector according to claim 15, wherein the hard part (30) of an injection-molded plastic and the diaphragm (31.1, 31.2) is made of a plastic film that can be welded to the hard part (30).

28. The high-pressure injector according to claim 14, wherein the connecting lines embodied in the control cassette (3) comprise a device (33) for separating gas bubbles from a liquid flowing through the connecting lines.

29. The high-pressure injector according to claim 14, wherein the control cassette (3) comprises an optical detection device (34) for detecting gas bubbles in a line segment (32.1, 32.2, 32.3, 32.4, 32.5, 32.6, 32.7, 32.8, 32.9).

30. The high-pressure injector according to claim 14, wherein in certain regions, the control cassette (3) is equipped with a heating device for the liquid that is conveyed through the connecting lines.

31. The high-pressure injector according to claim 14, wherein the control cassette (3) and at least one syringe (2.1, 2.2) are combined into an interchangeable unit and connected to each other and can be jointly inserted into the receiving chamber (110) and the syringe holder (10).

32. The high-pressure injector according to claim 14, wherein the receiving chamber (110) and/or the control cassette (3) is each equipped with a correctly positioned insertion of the control cassette (3) into the receiving chamber (110).

33. The high-pressure injector according to claim 14, wherein the control cassette (3) is embodied with an identification which is readable from the high-pressure injector when a control cassette (3) is inserted into the receiving chamber (110).
